(19) United States
(12) Patent Application Publication
(10) Pub. No.: US 2023/0210392 A1
(43) Pub. Date: Jul. 6, 2023

(54) Physiological Sensing Patch for Coupling a Device to a Body of a User

(71) Applicant: Apple Inc., Cupertino, CA (US)

(72) Inventors: Motohide Hatanaka, Menlo Park, CA (US); Albert Wang, Sunnyvale, CA (US); Wegene H. Tadele, San Francisco, CA (US)

(21) Appl. No.: 17/987,429

(22) Filed: Nov. 15, 2022

Related U.S. Application Data
(60) Provisional application No. 63/296,404, filed on Jan. 4, 2022.

(51) Int. Cl. A61B 5/024; A61B 5/0245; A61B 5/00

(52) U.S. Cl. CPC A61B 5/02438; A61B 5/0245; A61B 5/681; A61B 5/02416; A61B 5/02444; A61B 5/6833; A61B 2562/0266; A61B 2562/0271

(57) ABSTRACT

Embodiments are directed to a patch for coupling a watch body to a body of a user. The patch can include a substrate formed from a flexible material and an adhesive disposed over a surface of the substrate and configured to couple the patch to the body of the user. The patch can include a watch-mounting component disposed over a surface of the substrate and configured to couple the watch body to the patch. The patch can include one or more sensing elements, each having a terminal configured to contact the user, an interface element configured to interface with a watch sensing element of the watch body, and a conduit operably coupling the first terminal to the first interface element. The sensing elements can transmit signals to the watch body and the watch body can determine a physiological measurement of the user using the first and second signals.

### CROSS-REFERENCE TO RELATED APPLICATION(S)

This application is a nonprovisional patent application of and claims the benefit of U.S. Provisional Pat. Application No. 63/296,404, filed Jan. 4, 2022 and titled “Physiological Sensing Patch for Coupling a Device to a Body of a User,” the disclosure of which is hereby incorporated herein by reference in its entirety.

### FIELD

The described embodiments relate generally to devices and systems for performing physiological measurements. More particularly, the present embodiments relate to a device or system used to couple a smart device to the body of a user.

### BACKGROUND

Modern medical devices can be used to measure health parameters of a user or patient. However, many traditional medical devices may be too bulky or expensive to be used during normal daily activities. The systems and techniques described herein may be used to obtain physiological measurements without some of the drawbacks of some traditional medical devices.

### SUMMARY

Embodiments are directed to a patch for coupling a watch body to a body of a user. The patch can include a patch substrate formed from a flexible material, an adhesive disposed over a first surface of the patch substrate and configured to couple the patch to the body of the user and a watch-mounting component positioned at a second surface of the patch substrate and configured to couple the watch body to the patch. The patch can also include a first sensing element that includes a first terminal configured to contact the body of the user at a first location, a first interface element configured to interface with a first watch sensing element of the watch body and a first conduit operably coupling the first terminal to the first interface element. The patch may include a second sensing element that includes a second terminal configured to contact the body of the user at a second location, a second interface element configured to interface with a second watch sensing element of the watch body and a second conduit operably coupling the second terminal to the second interface element. The first and second sensing elements can be configured to transmit first and second signals to the watch body, respectively, and the watch body can be configured to determine a physiological measurement of the body using the first and second signals.

Embodiments are also directed to a patch for coupling a watch body to a body of a user that can include a patch substrate formed from a flexible material and an adhesive disposed over a first surface of the patch substrate and configured to couple the patch to the body of the user. The patch can include a watch-mounting component at a second surface of the patch substrate and configured to couple the watch body to the patch, where the watch body may have an optical sensing module configured to contact a skin surface of the user when the watch body is coupled to the patch. The patch can also include a sensing element that includes a terminal configured to contact the skin surface of the user and an interface element operably coupled to the terminal and configured to interface with a sensing module on the watch body.

Embodiments are further directed to a patch for coupling an electronic device to a user, where the patch includes a body segment, an adhesive positioned at a first side of the body segment and a coupling feature positioned at a second side of the body segment and configured to removably couple the patch to the electronic device. The patch can include a first electrical sensing assembly that includes a first conductive pad positioned at a first end of the body segment and configured to contact the user, a first conductive interface configured to contact a first electrical sensing element of the electronic device and a first signal conduit that couples the first conductive interface to the first conductive pad and is configured to transmit a first sensing signal. The patch can include a second electrical sensing assembly that includes a second conductive pad positioned at a second end of the body segment and configured to contact the user, a second conductive interface configured to contact a second electrical sensing element of the electronic device and a second signal conduit that couples the second conductive interface to the second conductive pad and is configured to transmit a second sensing signal.

### BRIEF DESCRIPTION OF THE DRAWINGS

The disclosure will be readily understood by the following detailed description in conjunction with the accompanying drawings, wherein like reference numerals designate like structural elements, and in which:

FIG. 1A shows an example patch coupling an electronic device to the body of a user;

FIG. 1B shows a bottom view of the electronic device when disconnected from the patch;

FIG. 1C shows the electronic device coupled to the user’s wrist using a watch strap;

FIG. 2 shows an example patch coupling an electronic device to a chest of a user;

FIG. 3A shows an example patch coupled to an electronic device;

FIG. 3B shows an exploded view of the example patch shown in FIG. 3A along with the electronic device;

FIG. 4 shows an example patch including an optical component;

FIG. 5 shows an example patch including a temperature sensor;

FIGS. 6A-6D show patches including different examples of coupling features for coupling a smartwatch to the patch;

FIG. 7 shows an example patch configuration;

FIG. 8 shows an example of a patch system that includes multiple patches for physiological sensing; and

FIG. 9 shows a block diagram of a physiological sensing system including a patch that couples a smartwatch to a user.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments disclosed herein are directed to a patch for coupling an electronic device, such as a smart device, to a body of a user. Smart devices, such as a smartwatch or a smartphone, are typically designed to be coupled to and/or interacted with by a user in a specific way. For example, a smartwatch typically includes a band that wraps around the wrist of the user to secure a watch body to the user. Smart devices can include one or more physiological sensors the measure and monitor physiological conditions of a user such as one or more cardiac parameters, respiratory information such as a breathing rate, body temperature, muscle function, and so on. However, the typical interaction between a user and the smart device may limit the amount, type or quality of physiological information that a smart device can measure. In some cases, physiological sensors, measurement processes and/or the physiological analysis may be designed to account for a specific type of interaction. For example, when sensing a user’s temperature using a smartwatch, an assumption may need to be made to estimate a user’s core temperature since the temperature sensing occurs at an extremity (e.g., a wrist) of the user.

In many cases, physiological sensors included in a smart device such as a smartwatch may be operated to determine a variety of physiological conditions for a user at different locations on a user’s body. However, this robust functionality of the smart device is often limited by the interaction of the user with the smart device. For example, a smartwatch is typically adapted to only be attached to a wrist of a user. Further, the results of many physiological measurements may be affected by the location of the sensor(s) and/or how the sensor is coupled to a user. For example, a smartwatch may take physiological measurements at the wrist, but the accuracy and/or sensitivity of these measurements may be increased by placing the smartwatch at other locations on a user. In some instances, the sensitivity and/or accuracy of an electrocardiogram (ECG) measurement on a smartwatch may be increased by placing the smartwatch body closer to a user’s heart and/or measuring the user over an extended time period. However, it can be impractical for a user to hold a smart device to their chest for an extended period of time. Moreover, a user may not be able to hold their smart device in a consistent or stable position for achieving a desirable measurement accuracy.

The devices and systems described herein include a patch that couples a smart device to the body of a user. The patch can be formed from a flexible material that contours to a user’s body and also includes an adhesive material that couples the patch to the user. The patch can be configured to expand the physiological sensing capabilities of a smart device by coupling the smart device to different locations on a user. Additionally, the patch can include sensing elements that interface with the smart device and contact the user to transmit physiological sensing data to the smart device.

In some examples, the smart device can include sensors that are operated to measure physiological parameters of a user. Example devices can include smartwatches, smartphones, tablets, wearable devices, mobile tracking devices, or other suitable device. As used herein, the term “physiological parameters” includes physiological information collected or measured about a user and can include information relating to functioning of one or more physiological systems such as cardiac function, body temperature, movement, posture, respiratory functions, muscle function, growth, sleep, stress, and/or any other suitable information about a user. In some cases, the smart device can measure and/or monitor physiological parameters of a user using sensors coupled to the smart device. Additionally or alternatively, the smart device may receive information from external sources such as additional devices that collect information about a user. For example, the smart device can receive information about a user’s weight, height, sleep, and so on from other devices that monitor and/or receive these types of information. In some cases, physiological information can be input into the smart device by the user.

The smart device can include a smartwatch that has one or more sensors for measuring physiological parameters of a user. The patch can be configured to couple a body of the smartwatch to locations on the user other than a user’s wrist. For example, a smartwatch typically is coupled to a user’s wrist by a band. Some smartwatches can include a watch body that detaches from the band and the watch body may include physiological sensors, a touch sensitive display, a battery and other electronics that support the functioning of the watch. As used herein the term “watch body” or “device body” may be used to refer to a device or portion of a device that is configured to attach to the wrist of a user by a band or strap. As used herein, the term “band” or “strap” includes bands/straps that include multiple components such as multiple band elements that couple together to attach a watch body to a user’s wrist. The patch can include a mounting component that couples the watch body to the patch. For example, the mounting components can interface with structures on the watch body that are used to couple the watch body to a band. In this regard, the patch can be used to couple the watch body to other locations on a user such as different regions on the user’s torso or other suitable locations. The patch can include a flexible substrate and an adhesive that couples to and may contour to a user’s body. For example, the patch can be substantially flat and conform to the contours of a user’s skin surface. In this regard, the patch may securely position one or more portions of the smartwatch against the skin surface of a user.

A smart device, such as a smartwatch, can include one or more sensors that contact the user to measure physiological parameters of the user. The patch can include sensing element that interphase with the sensors on the smartwatch and transmit physiological signals between a user’s body and the smartwatch. As used herein the term “smartwatch” is used to refer to a device that can be worn on a wrist and includes a touchscreen display and may have functionality that is adapted through the use of one or more applications or software programs that can be installed in internal memory and/or executed by an internal processor. The term “smartwatch” can also include wrist worn devices that have one or more integrated physiological sensors. For example, a smartwatch can have a heart rate monitor, an electrocardiogram (ECG) sensor, temperature sensors, respiratory sensors, other cardiac and/or pulmonary sensors, muscle and/or skin sensor systems, and/or the like.

In some embodiments an ECG sensor can include electrodes positioned on a back surface of the watch body and contact a user’s skin when the smartwatch is worn on a user’s wrist. When the watch body is coupled to other areas of the user, such as a user’s chest, it may be desirable to change the positioning of the electrode contact points with the user. For example, if using the patch to take an ECG measurement at the user’s chest, it may be desirable to increase a distance between the electrodes’ contact points with the user’s body. In this regard, the patch can include a first sensing element that interfaces with a first electrode on the watch body and changes a sensing location of the first electrode on the user’s body. The first sensing element can include a conductive terminal that contacts the body of the user at a location different from the location of the first electrode. The first sensing element can also include a conductive interface element that contacts the first electrode on the watch body and an electrical conduit that carries signals from the terminal and to the interface element where they are sensed by the first electrode on the watch body.

Additionally, the patch can include one or more additional sensing elements to interface with other electrodes on the watch body and can change a sensing location of these other electrodes. In this regard, the patch can change and/or expand the physiological sensing capabilities of sensors located on the watch body, such as an ECG sensor.

As another example, the patch can include a sensing element that changes the location of a temperature measurement taken by a smart device such as a smartwatch. In some cases, a smartwatch or other smart device can include a temperature sensor mounted to the body of the device. In these cases, heat generated from electrical components in the device body can affect body temperature measurements taken by sensors mounted on the watch body. To reduce these types of temperature effects on a body temperature measurement, the patch can include a sensing element that positions a temperature sensing terminal away from the device body. The temperature sensing terminal can contact a user and a conduit can carry temperature sensing signals from the temperature sensing terminal and to the watch body. In this regard, the patch may facilitate a body temperature sensing system that has greater isolation from other temperature sources such as electrical components of a smart device. The temperature sensing element can be implemented in a variety of ways including electrical temperature sensors (e.g., thermistors, thermocouples, and so on), optical temperatures sensors, or other suitable temperature sensors.

A smartwatch can also include one or more optical sensing system, which can be located on an underside of the device and contact the skin surface of the user while it is worn. The optical sensing system an transmit light into the user and receive a reflect portion of the transmitted light. The optical sensing system can determine physiological parameters based on the transmitted and received light. For example, the optical sensing system can be configured to make photoplethysmography (PPG) measurements.

In some cases, a smart device may include sensors that measure position, movement and/or other motion of a user. For example, a smart device such as a smartwatch can include one or more accelerometers, positioning devices (e.g., global positioning system (GPS) devices), other geolocation devices, relative positioning systems, gyros, magnetometers or other suitable positioning systems that can be used to determine position and/or orientation. The patch can be used to couple the smart device to a desired location on a user to track their movement parameters. For example, the patch may be used to couple the smart device to a user’s chest to monitor respiration parameters for the user. In this regard, the patch can enable a smart device such as a smartwatch to track physiological movement parameters such as a respiratory rate. In some cases, the smart device can combine this information with data from other sensors that are located on the smart device or received from other devices. For example, the smart watch may include a microphone that can monitor respiratory sounds of the user and respiratory sound data can be analyzed in coordination with the respiratory movement data. In some examples, the respiratory sound data can be collected from another device such as one or more earbuds.

In some cases, multiple patches can be used to coupled multiple devices to a user to perform physiological sensing at multiple locations on the user. In these cases, the data from the multiple devices may be combined to generate an integrated data set. For example, multiple devices can be used to determine a posture of a user, such as whether a user is standing up, laying down, bent over, and so on. In some cases, the multiple devices can track movements of a user, which can be used for a variety of purposes, including training, rehabilitation, diagnosis of conditions, efficiency analysis and so on. In some cases, multiple patches can be used to couple multiple smart devices to different locations on a user. In other examples, combinations of smart devices and more simple sensing devices may be used. For example, a first patch may couple a first smart device such as a smartwatch to a user and additional patches can be used to couple other simpler sensing devices to other parts of a user. The simpler sensing devices may include devices that perform dedicated sensing functions and therefore may be smaller, use less energy, and so on. In this regard, these simpler sensors may transmit physiological information to the smartwatch for analysis. Examples of these simpler sensors may include motion tracking devices, temperature tracking devices, ECG sensors, or other suitable devices. In this regard, the data from the multiple different sensors can be used to generate additional information about physiological parameters of a user.

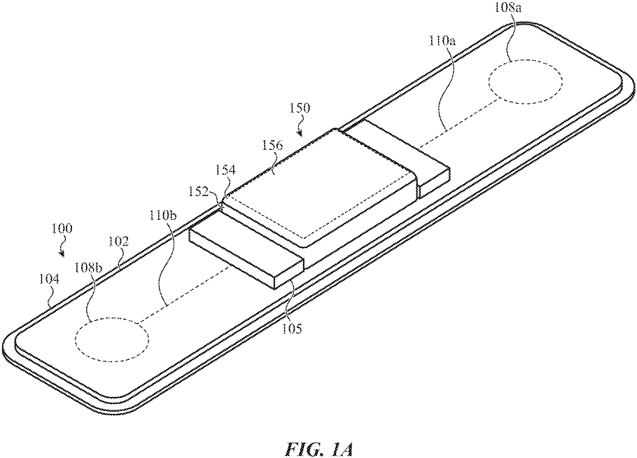

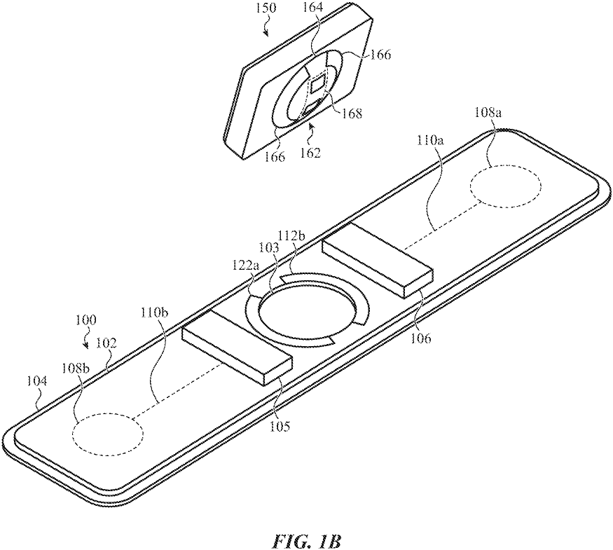

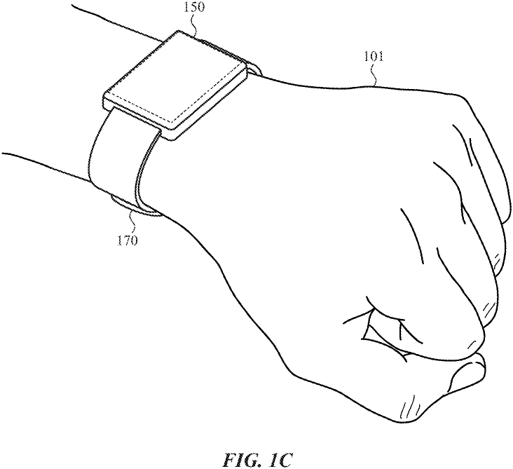

These and other embodiments are discussed below with reference to FIGS. 1A-9. However, those skilled in the art will readily appreciate that the detailed description given herein with respect to these Figures is for explanatory purposes only and should not be construed as limiting.

FIG. 1A shows a perspective view of an example patch 100 coupling an electronic device 150 a user. The patch 100 may include a substrate 102 that defines a body of the patch 100. The substrate 102 can be formed from a flexible material and be configured to conform, or otherwise deform to a skin surface of the user. The substrate 102 can include an adhesive 104 that couples the patch 100 to the user. The adhesive 104 can be located on a first side of the substrate 102 and can include any suitable adhesive for coupling to a user, such as a medical grade adhesive. In some cases, the adhesive 104 can be strong enough to securely hold the patch 100 and electronic device 150 to the user and also be removable by the user.

The patch 100 can include a mounting component 105 that couples the electronic device 150 to the patch 100. In some cases, the mounting component 105 can be attached to or formed as part of the substrate 102. The mounting component 105 can include structures that attach to features on the electronic device 150. For example, in cases where the electronic device 150 includes openings for accepting a band, the mounting component 105 can include structures that fit into these openings to couple the electronic device 150 to the user. In other examples, the electronic device 150 may include lugs or other features that are used to couple to a band and the mounting component 105 can be configured to couple with these lug features to secure the electronic device 150 to the patch 100, as described herein in greater detail.

The patch 100 can also include one or more sensing elements that are used to measure physiological parameters of the user. A sensing element can include a terminal 108 that contacts a skin surface of the user, a conduit 110 that carries signals to and/or from the terminal 108, and an interface element 112 (shown in FIG. 1B) that interfaces with a sensing element on the electronic device 150 and receives and/or transmits signals to the terminal 108. The signals that are transmitted between the terminal 108 and the electronic device 150 can be used to determine one or more physiological parameters of the user.

In some examples, the sensing elements can be configured to take an ECG measurement of the user. For example, a first sensing element can include a first terminal 108a that contacts a skin surface of the user at a first location and a second sensing element can include a second terminal 108b that contacts the skin surface of the user at a second location. The first and second terminals 108a, 108b can contact the user and be configured to detect electrical cardiac activity of the user. The first sensing element can include a first interface element 112a (shown in FIG. 1B) that interfaces with a sensing electrode on the electronic device 150 and a first conduit 110a that electrically couples the first terminal 108a to the first interface element 112a. The second sensing element can include a second interface element 112b (shown in FIG. 1B) that interfaces with a different sensing electrode on the electronic device 150 and a second conduit 110b the electrically couples the second terminal 108b to the second interface element 112b.

The electronic device 150 is depicted as a body of a smartwatch (e.g., a housing without the band attached, which may be referred to simply as a “smartwatch”), though this is one example embodiment of a smart device and the concepts discussed herein may apply equally and by analogy to other electronic devices, including health monitoring devices, fitness trackers, other wearable devices or the like. Other electronic devices including mobile phones (e.g., smartphones), digital media players (e.g., mp3 players), small tablets, trackers or tags, and other portable electronic devices may also be attached to a similar type of patch using the techniques described.

The electronic device 150 may include a housing 152 and a transparent cover 154 (which may be referred to simply as a “cover”) coupled with the housing 152 and positioned over a display 156. The cover 154 and the housing 152 along with other components may form a sealed internal volume of the electronic device 150, which may contain the internal electrical components of the electronic device 150. In some cases, the cover 154 defines substantially the entire front face and/or front surface of the electronic device 150. The cover 154 may also define an input surface. For example, as described herein, the electronic device 150 may include touch and/or force sensors that detect inputs applied to the cover 154. The cover 154 may be formed from or include glass, sapphire, a polymer, a dielectric, or any other suitable material.

The display 156 may be positioned under the cover 154 and at least partially within the housing 152. The display 156 may define an output region in which graphical outputs are displayed. Graphical outputs may include graphical user interfaces, user interface elements (e.g., buttons, sliders, etc.), text, lists, photographs, videos, or the like. The display 156 may include a liquid-crystal display (LCD), an organic light emitting diode display (OLED), or any other suitable components or display technology. In some cases, the display 156 may output a graphical user interface with one or more graphical objects that display information.

The display 156 may be touch- and/or force-sensitive and include or be associated with touch sensors and/or force sensors that extend along the output region of the display and which may use any suitable sensing elements and/or sensing techniques. Using touch sensors, the electronic device 150 may detect touch inputs applied to the cover 154, including detecting locations of touch inputs, motions of touch inputs (e.g., the speed, direction, or other parameters of a gesture applied to the cover 154), or the like. Using force sensors, the electronic device 150 may detect amounts or magnitudes of force associated with touch events applied to the cover 154. The touch and/or force sensors may detect various types of user inputs to control or modify the operation of the device, including taps, swipes, multiple finger inputs, single- or multiple-finger touch gestures, presses, and the like. Touch and/or force sensors usable with wearable electronic devices, such as the electronic device 150, are described below.

FIG. 1B shows a bottom view of the electronic device 150 when disconnected from the patch 100. The patch 100 can include one or more interface elements 112 that can receive physiological signals from one or more terminals 108. The interface elements 112 can be configured to interface with sensing elements or other interfaces on the electronic device 150.

In some cases, the electronic device 150 can include a sensor system 162 positioned on a back side of the electronic device 150 and configured to contact a skin surface of the user. The senor system 162 can include optical, electronic and/or other sensors that are configured to measure physiological parameters of a user such as heart rate, temperature, electrical cardiac activity, blood parameters, and so on. In some cases, the sensor system 162 can include sensors such as a photoplethysmography (PPG) sensor, electrocardiogram (ECG) sensor components, and/or the like.

In some cases, the sensor system 162 can include optical sensing components such as a PPG sensor that includes one or more light emitters and detectors 168. The optical sensing system 162 may include an optically transparent section 164 positioned on the back of the electronic device 150 which can allow light signals to be transmitted from and received by the sensing system 162. These light signals may be used to determine physiological parameters of a user such as heart rate, oxygen saturation, respiration, so on.

The patch 100 can define an opening 103 and the optical sensing system 162 can be positioned at least partially within the opening such that light signals can be transmitted and received through the opening 103. In some cases, the opening 103 can include bevels, tapers or other features that interface with the electronic device and facilitate contact of the optical sensing system 162 with the skin of the user.

Additionally or alternatively, the sensor system 162 can include an ECG sensor that has one or more electrodes 166 that can be positioned on a back side of the electronic device 150. In some cases, the electrodes 166 can be positioned on or around an optically transparent region 164 of an optical sensing system and contact a skin surface of the user when coupled to the patch 100. In other cases, the electrodes 166 can contact the interface elements 112 on the patch. For this reason, the terminals 108 may contact the user and transmit signals to the electronic device 150 using the connection of the interface elements 112 to the electrodes 166.

FIG. 1C shows the electronic device 150 coupled to the user’s 101 wrist using a watch band 170. The band 170 may be configured to couple the electronic device 150 to a user, such as to the user’s arm or wrist. A portion of the band 170 may be received in a band slot that is defined by a channel formed along a side of the housing 152. The band 170 may be secured to the housing 152 within the channel to maintain the band 170 to the housing 152.

When the electronic device 150 is not coupled to the user using the patch 100, it may be coupled to the user using the band 170. In this regard, the electronic device may monitor one or more physiological parameters of the user when attached to the wrist. In some cases, if an abnormal condition is detected at the wrist, the electronic device can instruct the user to place the electronic device 150 at a specific location of the user’s body using the patch 100. For example, if an abnormal heart rhythm is detected while the electronic device is being worn around the user’s wrist, then the electronic device 150 may instruct the user to attach the electronic device to the user’s 101 chest using the patch 100. This may allow the device to take measurements with greater sensitivity and/or accuracy to obtain further information about the physiological parameters of the user.

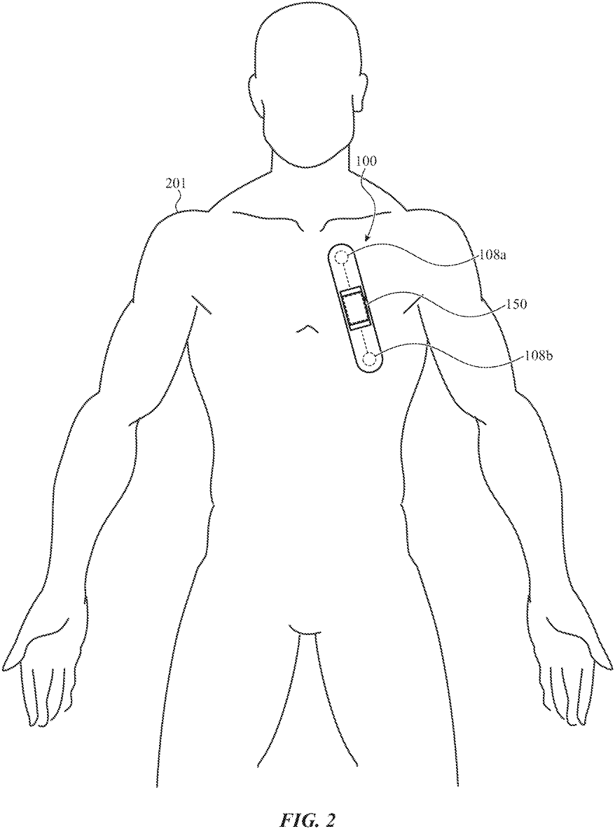

FIG. 2 shows the patch 100 coupling an electronic device 150 to a chest of a user 201. The sensing elements can be configured to measure electrical cardiac signals of the user 201. The patch 100 can be placed on the user 201 in a variety of orientations, which may facilitate a specific type of measurement. For example, the patch 100 can be coupled to a region of the user’s chest 201 where the heart is located. The first terminal 108a can contact the user 201 at a first location and the second terminal 108b can contact the user 201 at a second location. In some cases, the first and second terminals 108a, 108b can be configured to measure electrical cardiac activity of the user 201. The first and second terminals 108a, 108b can include conductive materials that can sense electrical changes in the user’s cardiac activity. In some cases, the first and second terminals 108a, 108b can include materials that improve electrical transmission from the user and to the terminals 108. For example, the terminals can include a gel contact, hydrogel, or other material that couples one or more conductive components to the user to detect the user’s cardiac signals.

The patch 100 can be configured in a variety of ways to position the terminals 108 at different spatial orientations with respect to each other and with respect to the user 201. For example, the electronic device 150 can include electrodes that have a first relative spacing. The patch 100 can be used to change this sensing orientation of the electrodes on the electronic device 150. For example, in some cases, it may be desirable to increase a distance between the electrodes to measure electrical cardiac changes over greater distances. In this regard, a distance between the first terminal 108a and the second terminal 108b can be greater than a distance between the electrodes located on the electronic device 150.

In some cases, an orientation of the terminals 108 can be configured for specific types of physiological sensing. For example, in the case of ECG sensing, it may be desirable to place the first terminal 108a near the base of the user’s 201 heart and the second terminal 108b near the apex of the heart. In this regard, the shape and spacing of the patch 100 can be configured to position the terminals 108 at these locations.

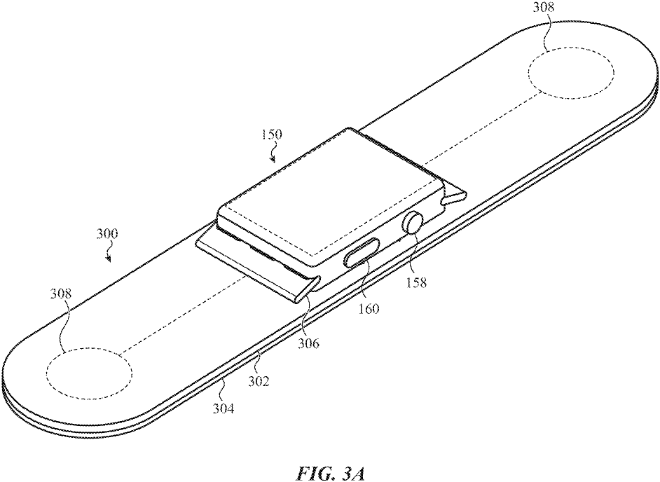

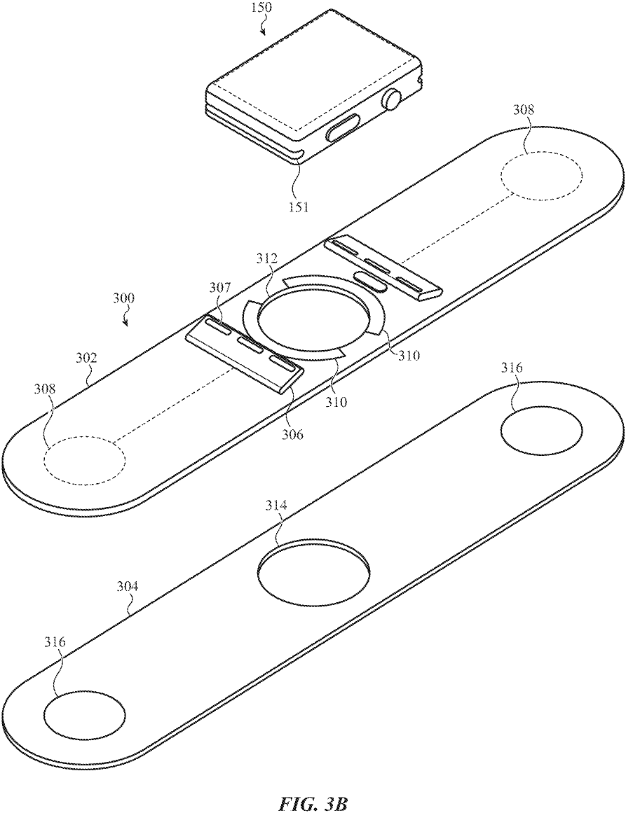

FIG. 3A shows an example patch 300 coupled to an electronic device 150. The patch 300 can be an example of the patches described herein. The patch 300 can include a substrate 302, an adhesive material 304 coupled to the substrate 302, a mounting component 306, and sensing elements 308 as described herein.

In some cases, the substrate 302 can be formed from a flexible material that conforms to the body of a user. For example, the substrate 302 can be formed from flexible polymer materials such as silicone materials, urethane materials, polyethylene, polypropylene, polyimides, and/or other suitable polymer materials. In other cases, the substrate 302 can include composite materials, which have multiple layers, integrated materials, areas with different material properties and so on. For example, a first portion of the substrate 302 can be formed from a rigid material and a second portion of the substrate 302 can be formed from a flexible material. In other cases, the substrate 302 can be formed from a rigid material such as metals, rigid polymers, rigid composite materials, or any other suitable material.

The adhesive material 304 can include medical grade adhesives that are configured to removably couple the substrate 302 to a user. The adhesive material 304 can be a layer that is disposed on a surface of the substrate 302 and can include liquid, gel and/or solid adhesives. In some cases, the adhesive material 304 can help facilitate physiological measurements by one or more sensors’ location on the patch 300 and/or the electronic device 150. For example, the adhesive material 304 can function as an electrical coupling material that helps pass electrical signals between a user and the sensing elements 308.

The mounting component 306 can be configured to couple to the electronic device 150 to secure the electronic device against the body of a user. The mounting component 306 can take on a variety of configurations as described herein. For example, the electronic device 150 can include band attachment features 151 that are used to couple the electronic device 150 to a band or other component. The mounting component 306 can be configured to engage with the band attachment features 151 to secure the electronic device 150 to the patch 300. In some cases, the mounting components 306 can include an elastic material that helps maintain contact between the electronic device 150 and a user. For example, the elastic material of the mounting component can flex or otherwise deform to accommodate movement of a user while maintaining contact between the electronic device 150 and the user.

In some cases, the mounting component 306 can include one or more communication interfaces 307, which can be operably coupled to one or more sensing elements 308. For example, the communication interface 307 can be an electrical interface that transmits electrical signals to the sensing element 308 and/or receives electrical signals from the sensing element. The communication interface(s) 307 can operably couple with interfaces on the electronic device 150, which can include interfaces located within the band attachment feature 151. For example, these interfaces can carry signals between the electronic device 150 and sensing elements 308 such as ECG terminals, temperatures sensors, or any other suitable sensor.

The patch 300 can include one or more sensing elements 308 that are configured to transmit physiological signals to the electronic device 150. The sensing elements 308 can include electrical sensing elements, which may measure cardiac electrical activity of a user; temperature sensors that measure a body temperature of the user; motion/position sensing components; respiratory sensors; sound monitoring; or any other suitable types of sensors.

The electronic device 150 may also include a crown 158 having a cap, protruding portion, or component(s) or feature(s) (collectively referred to herein as a “body”) positioned along a side surface of the housing 152. At least a portion of the crown 158 (such as the body) may protrude from, or otherwise be located outside, the housing 152, and may define a generally circular shape or circular exterior surface. The exterior surface of the body of the crown 158 may be textured, knurled, grooved, or otherwise have features that may improve the tactile feel of the crown 158 and/or facilitate rotation sensing.

The electronic device 150 may include a crown 158 that facilitates a variety of potential interactions. For example, the crown 158 may be rotated by a user (e.g., the crown may receive rotational inputs). Rotational inputs of the crown 158 may zoom, scroll, rotate, or otherwise manipulate a user interface or other object displayed on the display 156 (among other possible functions). The crown 158 may also be translated or pressed (e.g., axially) by the user. Translational or axial inputs may select highlighted objects or icons, cause a user interface to return to a previous menu or display, or activate or deactivate functions (among other possible functions).

The electronic device 150 may also include other inputs, switches, buttons, or the like. For example, the electronic device 150 includes a button 160. The button 160 may be a movable button (as depicted) or a touch-sensitive region of the housing 152. The button 160 may control various aspects of the electronic device 150. For example, the button 160 may be used to select icons, items, or other objects displayed on the display 156, to activate or deactivate functions (e.g., to silence an alarm or alert), or the like.

FIG. 3B shows an exploded view of the example patch 300 and the electronic device 150. In some cases, the patch 300 can include one or more interface elements 310 that contact a sensing system on the electronic device 150 and carry signals between the sensing elements and the electronic device 150. In some cases, the interface elements 310 can include conductive materials that are coupled to the substrate 302. Additionally or alternatively, the interface elements 310 may include optical components such as fiber optics that carry optical signals.

In some cases, the substrate 302 can define one or more openings that correspond to sensor systems of the electronic device 150. For example, the substrate 302 can include an opening 312 that aligns with an optical system of the electronic device 150. The opening can be configured such that an optical window or other structure on the electronic device 150 contacts a skin surface of a user and/or can transmit and receive light signals while coupled with the patch 300. In some cases, the adhesive material 304 can also include an opening 314 for optical sensing components.

In some cases, the adhesive material 304 can be adhesives that are adapted for removably coupling to human skin. the adhesive material 304 can include a pressure sensitive adhesive layer that is disposed on a bottom surface of the substrate 302. The adhesive material can be conductive such that electrical signals can be sensed by the sensing elements 308 through the adhesive material. In other cases, the adhesive material 304 can include regions 316 that correspond to sensing elements 308 of the patch 300. For example, the regions 316 can include a gel contact, or other electrical coupling material that is positioned between the sensing elements 308 and the skin of the user. In some cases, the sensing elements include terminals, as described herein, and the terminals can include a gel material that contacts the user, which can be at one or more of the regions 316. In cases of temperature sensing, the regions 316 may include thermal coupling materials, which could include material that isolates temperature sensing components from the external environment to help improve the accuracy of body temperature measurements.

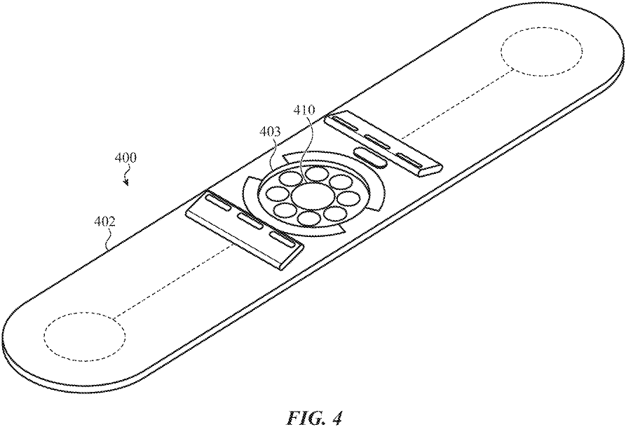

FIG. 4 shows an example patch 400 including an optical component 410. The patch 400 can be an example of the patches described herein. The patch 400 can include a substrate 402 that defines the body of the patch. The substrate 402 can define an opening 403 for optical components on an electronic device, as described herein. In some cases, the patch can include the optical component 410 in the opening 403. The optical component 410 can be configured to change a direction of emitted or reflected light, focus the light, or otherwise modify the light paths to aid optical sensing by the electronic device. In some cases, the optical component 410 can include one or more lens elements or components that focus that light such as a Fresnel lens, collimating lens, optical filters, or any other suitable component. In some cases, the optical component 410 can have different regions that perform different functions. For example, a peripheral region can block specific wavelengths of light and a central region that can allow the blocked wavelengths to pass. In some cases, the optical component 410 can include a banded fiber-optic component.

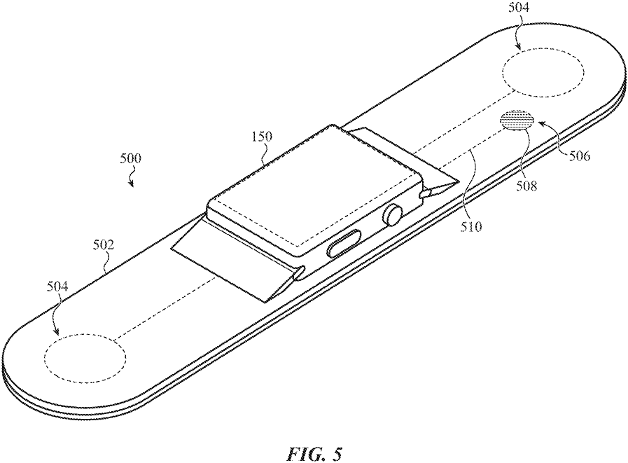

FIG. 5 shows an example patch 500 including a temperature sensor. The patch 500 can be an example of the patches described herein and include a substrate 502 that defines a body of the patch 500. The patch can include one or more sensing elements 504, 506 that perform different sensing functions. For example, sensing elements 504 can include ECG sensing terminals as described herein and the sensing element 506 can be a temperature sensing element. In some cases, the patch 500 may include only one type of sensing element such as a temperature sensing element 506 or ECG sensing elements 504.

The temperature sensing element 506 can include a terminal 508 that contacts that body of a user and a conduit 510 that carries signals between the electronic device 150 and the terminal 508. In some cases, the terminal 508 can be positioned away from the electronic device 150 to reduce thermal effects from heat generated by the electronic device 150. In this regard, the substate can thermally isolate the terminal 508 from the electronic device, and/or the external environment.

The temperature sensing element 506 can include electrical temperature sensors such as thermopiles, thermocouples, or any other suitable temperature sensing system. In these cases, the conduit 510 can receive temperature signals from the terminal 508 and transmit those signals to the electronic device 150. In some cases, the temperature sensing element 506 can be configured to generate a signal that corresponds to a temperature of the skin surface. The signal can be an electrical signal that varies in accordance with the temperature or the skin surface and can be transmitted to the electronic device 150 via an interface element, as described herein. In other cases, the signal can be an optical signal that varies in accordance with the temperature of the skin surface of a user and can be transmitted to the electronic device 150 via an interface element or other structure (e.g., optical fiber(s)), as described herein.

The temperature sensing element 506 can include optical temperature sensors. In some cases, this can include using infrared temperature sensors to measure the body temperature of a user. For example, the terminal 508 can comprise an optical coupled that transmits and/or receive optical (e.g., infrared signals) from the skin surface of a user, and the conduit 510 can include optical components such as fiber optics that carry the optical signals to and from the electronic device 150. In other cases, a color-changing temperature sensing film can be coupled to the user, and an optical sensor can determine the user’s body temperature based on color changes in the optical film. In other examples, acoustic temperature sensing may be performed, for example, using a temperature sensing waveguide that changes an acoustic transmission rate in response to changes in temperature.

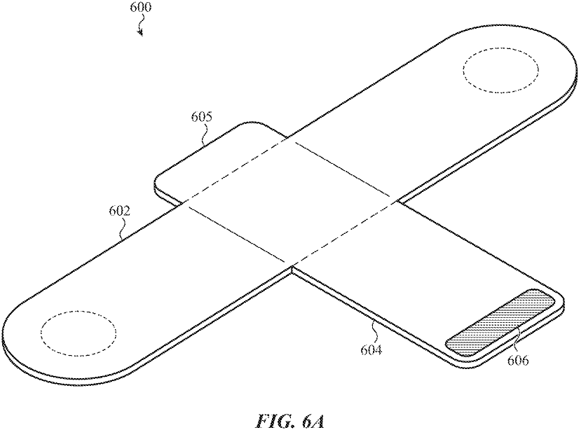

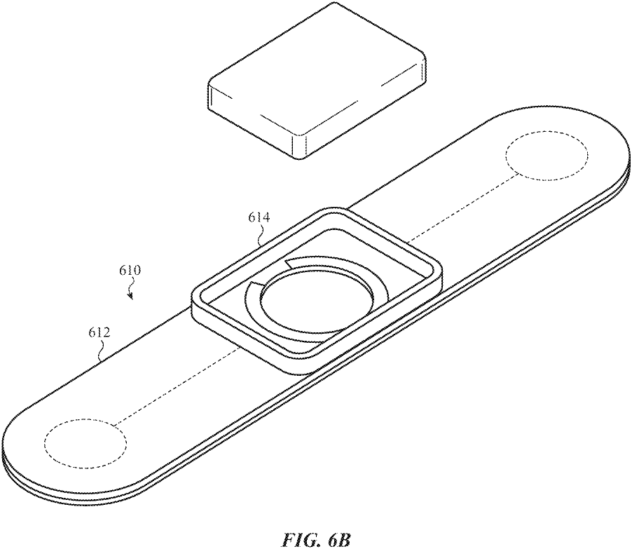

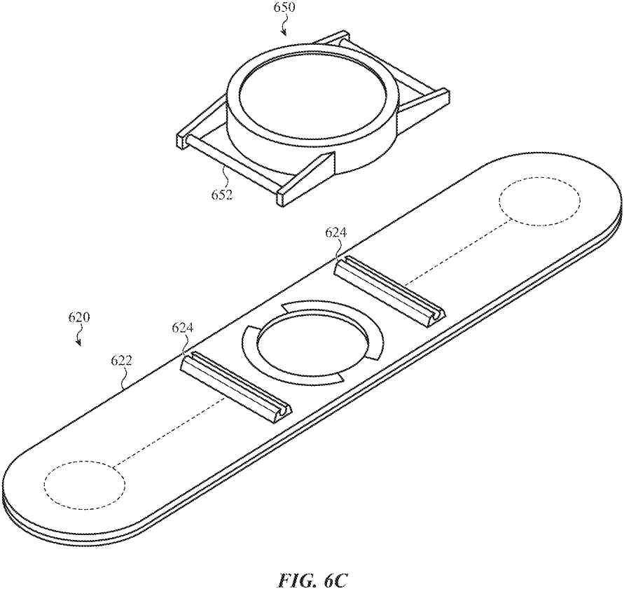

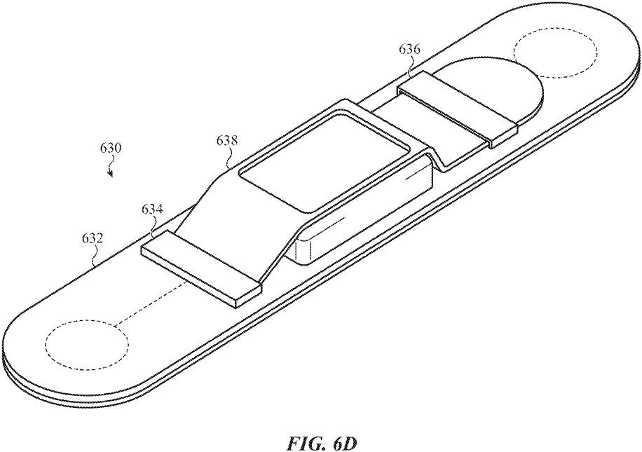

FIGS. 6A-6D show patches including different examples of coupling features for coupling the electronic device 150 to the patch. In a first example shown in FIG. 6A, patch 600 can include a substrate 602 that defines a tab 604 that folds over to a body region 605 of the substrate. In this regard, the electronic device 150 can be placed on the body region 605 and the tab 604 can be folded over the electronic device 150 to couple the electronic device to the patch 600. The patch 600 can also include an adhesive material on a bottom surface that couples the patch 600 to a user. In some cases, the tab 604 can include a coupling feature 606 such as an adhesive that couples the tab 604 to the body region 605 to secure the electronic device 150 in place.

In another example shown in FIG. 6B, the patch 610 can include a substrate 612 and a shell 614 that is coupled to the substrate 612. The electronic device 150 can be inserted into the shell 614 and the shell 614 can couple the electronic device to the patch 610. In some cases, the shell 614 can be formed from a flexible material and wrap around or conform to one or more portions of the electronic device. In other cases, the shell 614 may include snap features, overhangs, or other features that secure the electronic device 150 to the patch 610.

In another example shown in FIG. 6C, the patch 620 can include mounting components 624 that are configured to couple with lugs on a watch body 650. For example, some watches 650 include lugs or bars 652 that slide through openings in band straps. The mounting components 624 can include openings that couple to these types of lugs 652 to secure the watch device to the patch 620. In some cases, the mounting components 624 can include snap and/or interference features that couple to watch lugs 652. In other examples, the lugs 652 can be inserted into the mounting components 624 to couple a watch device to the patch 620.

In another example shown in FIG. 6D, the patch 630 can include a strap 638 that extends over the top of the electronic device 150 to secure the electronic device to the patch 630. The patch 630 can include a substrate 632 that defines a body of the patch 630 and the strap 638 can couple to the substrate 632 at a first location 634. The strap 638 can include a fastening feature 636 at the other end that secures the strap 638 in place over the electronic device. In some cases, the strap 638 can include an opening for a display of the electronic device 150. The strap 638 can be formed from a flexible material such that it securely couples the electronic device 150 to the patch 630.

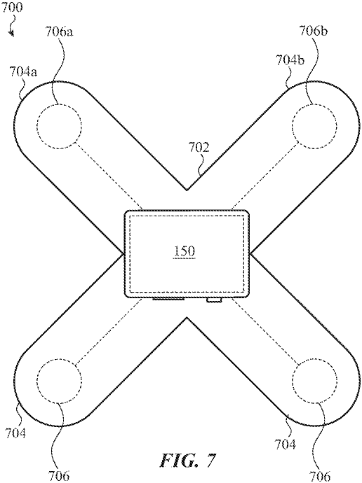

FIG. 7 shoes an example of a patch 700 that includes multiple sensing regions 704. The patch 700 can be an example of the patches described herein and be configured to couple the electronic device 150 to user as described herein. The sensing regions 704 can each include one or more sensing elements 706 that measure a physiological parameter of a user. In some cases, the sensing elements 706 can function in coordination to take a physiological element, for example by each functioning as an electrode for an ECG measurement. In other cases, different ones of the sensing elements 706 can measure different physiological parameters. For example, different sensing regions 704 can include different types of physiological sensors.

In some cases, the sensing regions can be configured to have different structures, which can be based on the type of physiological measurement. For example, a first sensing region 704a can position a first sensing element 706a further away from the electronic device than other sensing regions 704. This can be beneficial for certain types of physiological measurements such as an ECG measurement, body temperature measurement, and/o the like. In other cases different sensing elements 706 can have different structures. For example, the first sensing element 706a can be configured to take an ECG measurement and may include a conductive element that is coupled to the user, as described herein. A second sensing element 706b can be configured to take an optical measurement and may include an optical coupler that contacts the user and transmits optical signals to the electronic device 150, as described herein.

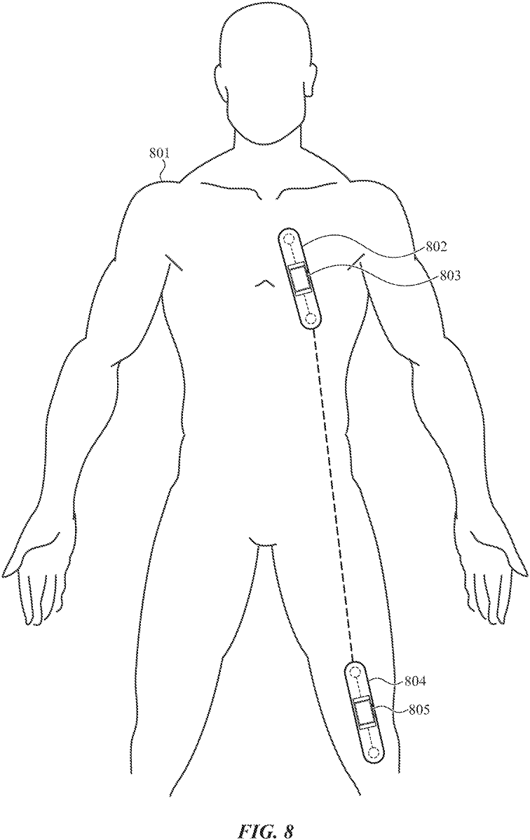

FIG. 8 shows an example of a patch system 800 that utilizes multiple patches for performing physiological sensing. The system 800 can include a first patch 802 coupled to a first position on a user 801 and a second patch 804 coupled to a second position on the user 801. The first patch 802 can couple a first electronic device 803 to the user 801 and the second patch 804 can couple a second electronic device 805 to the user 801. The first and second patches 802, 804 can be examples of the patches described herein, and the electronic devices 803, 805 can be examples of the electronic devices described herein. The first and second electronic devices 803, 805 can establish a communication connection using any suitable technology such as Bluetooth, Wi-Fi, or any other suitable wired or wireless communications protocols. The first and second electronic devices 803, 805 can coordinate physiological sensing activities, transmit data and exchange other information over the communication connection.

In some cases, the first and second electronic devices 803, 805 can be a same type of device such as a smartwatch, smartphone, or other physiological sensing device. In some cases, the first electronic device 803 can be a first type of device such as a smart device and the second electronic device 805 can be a second type of device, which may have less functionality. For example, the second type of device may not have a touch-display, as powerful of electronic processing components, or as much functionality as the first electronic device 803. In this regard, the second electronic device 805 may be a dedicated sensor that is configured to perform one or more types of measurements. For example, the second electronic device 805 may be a tracking device that can be used to track the position and/or movement of the user 801. In this regard, the second electronic device 805 may measure one or more parameters and send data to the first electronic device 803 for further processing.

In some cases, the second electronic device 805 may include components such as a battery, signal processing electronics, signal transmission covenants such as one or more antennas for transmitting wireless signals and one or more physiological sensors.

In some examples, the first and second electronic devices 803, 805 may be operated to perform ECG measurements with additional leads as compared to a single device. Although only two electronic devices are shown, additional electronic devices can be coupled to other locations of the user to obtain additional physiological data such as additional ECG leads.

In other cases, multiple electronic devices can be coupled to the user 801 to perform position, posture, and/or movement analysis. The multiple devices can track movements of a user, which can be used for a variety of purposes, including training, rehabilitation, diagnosis of conditions, efficiency analysis and so on. In some cases, multiple patches can be used to couple multiple smart devices to different locations on a user. Position, posture, movement tracking and/or analysis may be performed using wireless signal localization of one or more of the electronic devices (e.g., electronic devices 803 and 805) with respect to each other. In some cases, a time-of-flight sensor can measure distance between different electronic devices using a radio frequency pulse or series of radio frequency pulses. Additionally or alternatively, one or more of the electronic devices 803, 805 can include multiple antennas positioned at different locations at each device. Distances between the different antenna can be used to determine an orientation and or position of each device relative to each other. For example, ultra-wide band signals can be used to determine an orientation of multiple components of each device relative to each other. This technology may be used to determine the position, movement, or other tracking of the user 801.

The electronic devices 803 and 805 can be operated to perform blood flow measurements such as pulse wave analysis. In some cases, one or more of the electronic devices 803, 805 can include sensors that measure pulse wave amplitude (PWA) of the user 801, which may be used to derive one or more blood flow metrics such a vasoconstriction metric. In some cases, the PWA can be detected using an optical sensing unit such as a PPG sensor. Additionally or alternatively, the electronic devices 803, 805 can be operated to measure a pulse wave velocity (PWV) of the user 801, which can be used to determine physiological metrics such as arterial stiffness. For example, the first electronic device 803 can be positioned at a chest area of the user 801 and the second electronic device 805 can be positioned at a leg and/or arm of the user 801. The electronic devices 803, 805 can be operated to measure the time it takes for a pulse wave to propagate from the first electronic device 803 and to the second electronic device 805. Ultra-wide band signals, microwave signals, radio frequency signals, and/or the like can be used to determine a distance and/or positioning of the electronic devices 803, 805, which can be used to determine the PWA and/or PWV of the user 801.

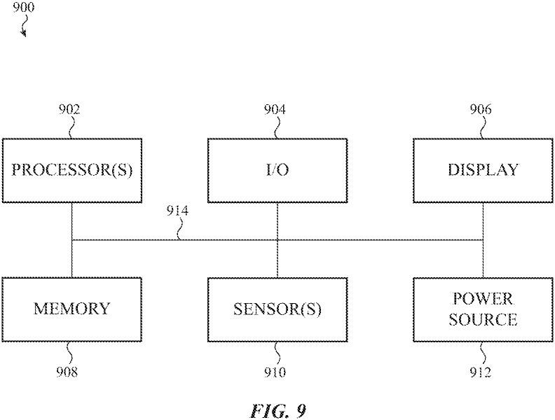

FIG. 9 is an example block diagram of a physiological monitoring device 900, which can take the form of any of the devices as described with references to FIGS. 1-8. The physiological monitoring device 900 can include a processor 902, an input/output (I/O) mechanism 904 (e.g., wired or wireless communications interfaces), a display 906, memory 908, sensors 910 (e.g., physiological sensors such as those described herein), and a power source 912 (e.g., a rechargeable battery). The processor 902 can control some or all of the operations of the physiological monitoring device 900. The processor 902 can communicate, either directly or indirectly, with some or all of the components of the physiological monitoring device 900. For example, a system bus or other communication mechanism 914 can provide communication between the processor 902, the I/O mechanism 904, the memory 908, the sensors 910, and the power source 912.

The processor 902 can be implemented as any electronic device capable of processing, receiving, or transmitting data or instructions. For example, the processor 902 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements.

It should be noted that the components of the physiological monitoring device 900 can be controlled by multiple processors. For example, select components of the physiological monitoring device 900 (e.g., a sensor 910) may be controlled by a first processor and other components of the physiological monitoring device 900 (e.g., the I/O 904) may be controlled by a second processor, where the first and second processors may or may not be in communication with each other.

The I/O device 904 can transmit and/or receive data from a user or another electronic device. An I/O device can transmit electronic signals via a communications network, such as a wireless and/or wired network connection. Examples of wireless and wired network connections include, but are not limited to, cellular, Wi-Fi, Bluetooth, IR, and Ethernet connections. In some cases, the I/O device 904 can communicate with an external electronic device, such as a smartphone, electronic device or other portable electronic device, as described here.

The physiological monitoring device may optionally include a display 906 such as a liquid-crystal display (LCD), an organic light emitting diode (OLED) display, a light emitting diode (LED) display, or the like. If the display 906 is an LCD, the display 906 may also include a backlight component that can be controlled to provide variable levels of display brightness. If the display 906 is an OLED or LED type display, the brightness of the display 906 may be controlled by modifying the electrical signals that are provided to display elements. The display 906 may correspond to any of the displays shown or described herein.

The memory 908 can store electronic data that can be used by the physiological monitoring device 900. For example, the memory 908 can store electrical data or content such as, for example, audio and video files, documents and applications, device settings and user preferences, timing signals, control signals, and data structures or databases. The memory 908 can be configured as any type of memory. By way of example only, the memory 908 can be implemented as random access memory, read-only memory, Flash memory, removable memory, other types of storage elements, or combinations of such devices.

The physiological monitoring device 900 may also include one or more sensors 910 positioned almost anywhere on the physiological monitoring device 900. The sensor(s) 910 can be configured to sense one or more types of parameters, such as but not limited to, pressure, light, touch, heat, movement, relative motion, biometric data (e.g., biological parameters), and so on. For example, the sensor(s) 910 may include a heat sensor, a position sensor, a light or optical sensor, an accelerometer, a pressure transducer, a gyroscope, a magnetometer, a health monitoring sensor, and so on. Additionally, the one or more sensors 910 can utilize any suitable sensing technology, including, but not limited to, capacitive, ultrasonic, resistive, optical, ultrasound, piezoelectric, and thermal sensing technology.

The power source 912 can be implemented with any device capable of providing energy to the physiological monitoring device 900. For example, the power source 912 may be one or more batteries or rechargeable batteries. Additionally or alternatively, the power source 912 can be a power connector or power cord that connects the physiological monitoring device 900 to another power source, such as a wall outlet.

As described above, one aspect of the present technology is determining physiological parameters of a user. The present disclosure contemplates that in some instances this gathered data may include personal information data that uniquely identifies or can be used to contact or locate a specific person. Such personal information data can include demographic data, location-based data, telephone numbers, email addresses, twitter IDs (or other social media aliases or handles), home addresses, data or records relating to a user’s health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide haptic or audiovisual outputs that are tailored to the user. Further, other uses for personal information data that benefit the user are also contemplated by the present disclosure. For instance, health and fitness data may be used to provide insights into a user’s general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user’s privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A patch for coupling a watch body to a body of a user, the patch comprising:
a patch substrate formed from a flexible material;
an adhesive disposed over a first surface of the patch substrate and configured to couple the patch to the body of the user;
a watch-mounting component positioned at a second surface of the patch substrate and comprising:
a first mounting structure configured to couple to a first band feature on the watch body;and
a second mounting structure configured to couple to a second band feature on the watch body;
a first sensing element comprising:
a first terminal configured to contact the body of the user at a first location;
a first interface element configured to interface with a first watch sensing element of the watch body; and
a first conduit operably coupling the first terminal to the first interface element; and
a second sensing element comprising:
a second terminal configured to contact the body of the user at a second location;
a second interface element configured to interface with a second watch sensing element of the watch body; and
a second conduit operably coupling the second terminal to the second interface element, the first and second sensing elements configured to transmit first and second signals to the watch body, respectively, the watch body configured to determine a physiological measurement of the user using the first and second signals.

2. The patch of claim 1, wherein:
the first sensing element comprises a first electrical sensing element configured to measure electrical cardiac activity of the user;
the first signal corresponds to the electrical cardiac activity, as measured at the first location;
the second sensing element comprises a second electrical sensing element configured to measure the electrical cardiac activity of the user; and
the second signal corresponds to the electrical cardiac activity, as measured at the second location.

3. The patch of claim 2, wherein:
the first watch sensing element comprises a first electrode;
the first interface element comprises a first conductive material configured to transmit the first signal to the first electrode;
the second watch sensing element comprises a second electrode; and
the second interface element comprises a second conductive material configured to transmit the second signal to the second electrode.

4. The patch of claim 2, wherein a distance between the first terminal and the second terminal is greater than a distance between the first watch sensing element and the second watch sensing element.

5. The patch of claim 1, wherein:
the first terminal comprises a first conductive gel; and
the second terminal comprises a second conductive gel.

6. The patch of claim 1, wherein:
the patch substrate defines an opening; and
an optical sensing unit of the watch body is positioned at least partially within the opening and is configured to obtain an optical measurement of the body of the user.

7. The patch of claim 6, wherein the patch further comprises an optical component positioned between the body of the user and the optical sensing unit.

8. The patch of claim 1, wherein the watch-mounting component comprises a tab that is configured to couple with a band slot of the watch body.

9. A patch for coupling a watch body to a body of a user, the patch comprising:
a patch substrate formed from a flexible material and defining an opening;
an adhesive disposed over a first surface of the patch substrate and configured to couple the patch to the body of the user;
a watch-mounting component at a second surface of the patch substrate and configured to couple the watch body to the patch, the watch body having an optical sensing module positioned at least partially within the opening and configured to contact a skin surface of the user and measure a physiological parameter of the user when the watch body is coupled to the patch; and
a sensing element comprising:
a terminal configured to contact the skin surface of the user; and
an interface element operably coupled to the terminal and configured to interface with a sensing module on the watch body.

10. The patch of claim 9, wherein:
the sensing module comprises a temperature sensor; and
the sensing element is configured to generate a signal that corresponds to a temperature of the skin surface.

11. The patch of claim 10, wherein:
the signal is an electrical signal that varies in accordance with the temperature of the skin surface;
the interface element is configured to transmit the electrical signal to the watch body; and
the temperature sensor is configured to estimate the temperature of the skin surface using the electrical signal.

12. The patch of claim 10, wherein:
the signal is an optical signal that varies in accordance with the temperature of the skin surface;
the interface element is configured to transmit the optical signal to the watch body; and
the temperature sensor is configured to estimate the temperature of the skin surface using the optical signal.

13. The patch of claim 9, further comprising an optical component operable to transmit emitted optical signals from the optical sensing module to the body and transmit reflected optical signals from the body to the optical sensing module.

14. The patch of claim 9, wherein the patch substrate is configured to couple the watch body to a torso portion of the user.

15. The patch of claim 9, wherein:
the watch-mounting component comprises an electrical interface; and
the sensing element is electrically coupled to the electrical interface and configured to transmit sensing signals to the watch body via the electrical interface.

16. A patch for coupling an electronic device to a user, the patch comprising:
a body segment;
an adhesive positioned at a first side of the body segment; and
a coupling feature positioned at a second side of the body segment and configured to removably couple the patch to the electronic device;
a first electrical sensing assembly comprising:
a first conductive pad positioned at a first end of the body segment and configured to contact the user;
a first conductive interface configured to contact a first electrical sensing element of the electronic device; and
a first signal conduit that couples the first conductive interface to the first conductive pad and is configured to transmit a first sensing signal;
a second electrical sensing assembly comprising;
a second conductive pad positioned at a second end of the body segment and configured to contact the user;
a second conductive interface configured to contact a second electrical sensing element of the electronic device; and
a second signal conduit that couples the second conductive interface to the second conductive pad and is configured to transmit a second sensing signal; and
a temperature sensing assembly comprising a temperature sensing lead configured to contact the user.

17. The patch of claim 16, wherein:
the first sensing signal is a first voltage signal;
the second sensing signal is a second voltage signal; and
the electronic device is configured to determine a physiological parameter of the user based on the first and second voltage signals.

18. The patch of claim 16, wherein:
the body segment defines an opening; and
an optical sensing unit of the electronic device is positioned at least partially within the opening when the electronic device is attached to the coupling feature.

19. The patch of claim 18, wherein:
when the electronic device is attached to the coupling feature, the optical sensing unit is configured to emit and receive light through the opening; and
the electronic device is configured to determine a physiological parameter of the user based on the emitted and received light.

20. The patch of claim 16, wherein:
the temperature sensing assembly is configured to output a temperature sensing signal to the electronic device; and
the electronic device is configured to determine a physiological parameter of the user based on the temperature sensing signal.
